(19) United States
(12) Patent Application Publication
(10) Pub. No.: US 2011/0028827 A1
(43) Pub. Date: Feb. 3, 2011

(54) SPATIOTEMPORAL PATTERN CLASSIFICATION OF BRAIN STATES

(76) Inventors: Ranganatha SITARAM, Tuebingen (DE); Niels BIRBAUMER, Moessingen (DE)

Correspondence Address: PILLSBURY WINTHROP SHAW PITTMAN, LLP, P.O. BOX 10500, MCLEAN, VA, 22102, US

(21) Appl. No.: 12/510,755

(22) Filed: Jul. 28, 2009

(51) Int. Cl. A61B 5/055; G06K 9/00

(52) U.S. Cl. 600/410; 382/131

(57) ABSTRACT

A multivariate, pattern-based system and method for recognition of brain states and generation of feedback considering both the spatial and temporal pattern of network of brain activity is described. The system can be applied for enhancing a desired function or behavior, or to alleviate a behavioral or neurological problem. The system can also be used to investigate the spatiotemporal evolution of a network of brain activity corresponding to a brain function, by changing the activity as an independent variable and studying its effect on behavior.

### BACKGROUND

Brain-Computer-Interfaces (BCI) enable control of computers and of external devices with regulation of brain activity alone. Two different traditions of BCI research have developed in the field: invasive BCI based on animal studies and realized with implanted electrodes, and non-invasive BCI, primarily using Electroencephalography (EEG), magnetic resonance imaging (MRI) techniques such as functional MRI (EVIRI) and Near Infrared Spectroscopy (NIRS).

Subjects can learn voluntary self-regulation of localized brain regions using signal or feedback from changes in their neural activity. The self-regulation training can be used for behavioural modifications of the subjects. If the neurobiological basis of a disorder (e.g., chronic pain, motor diseases, psychopathy, social phobia, depression) is known in terms of abnormal activity in certain regions of the brain, the BCI can be targeted to modify activity in those regions with high specificity for treatment.

### BRIEF SUMMARY

The disclosure describes, among other information, various apparatus, systems and methods of use relating to a real-time spatiotemporal pattern classifier and associated read-out optionally including both spatial and temporal spectroscopic data.

### BRIEF DESCRIPTION OF THE DRAWINGS

The following drawings form part of the present specification and are included to further demonstrate, but not to limit, certain embodiments. The specification and claims may be better understood by reference to one or more of these drawings in combination with the detailed description of specific embodiments presented herein.

FIG. 1: shows optional architecture of an illustrative embodiment of an fMRI-BCI based on spatiotemporal pattern classification of brain states.

FIG. 2: shows optional embodiments of the use of spatio-temporal information for input to a pattern classifier. FIG. 2(a) depicts the spatio-temporal information from the brain and FIG. 2(b) depicts the sliding window approach to reducing the spatio-temporal dimension of data.

FIG. 3: shows optional embodiments of the use of Hidden Markov Model (HMM) for real-time spatiotemporal classification of brain states in an fMRI-BCI. FIG. 3(a) depicts the vector representation of fMRI data for each time point t and FIG. 3(b) depicts the Markov Modern M for each label.

FIG. 4: shows an illustrative embodiment of the HMM method of training and testing to classify brain states using fMRI signals.

FIG. 5: shows optional embodiments of the illustrative HMM method of training and testing of FIG. 4. Each run has 3 blocks of emotional picture presentation alternating with a fixation cross. Six pictures are presented in each block of emotion. Each picture will be shown for 6 s. Students were asked to attend to each picture without moving their head and body. During fixation block students were requested to count back from 100 to 1.

FIG. 6: shows optional embodiments of HMM method of training and testing of FIG. 4. Each run has 3 blocks of emotional recall instructions alternating with a fixation cross. Instructions were given in English and Gelman. In each recall block students were instructed to recall corresponding emotions (neutral, happy and disgust). During fixation block students were requested to count back from 100 to 1.

FIG. 7: shows an illustrative embodiment of a graphical thermometer that provides real-time feedback of the emotional state detected by multivariate SVM.

FIG. 8: shows an illustrative embodiment of a readout of the performance of the brain state classifier.

FIG. 9: shows illustrative embodiments of the differences in brain activation as obtained by the conventional univariate GLM analysis and the multivariate SVM analysis. Generally, with GLM maps, yellow/red voxels show significant activations and blue/green voxels show significant deactivations at the given contrast (e.g., Happy v. Neutral). With the SVM maps, yellow/red voxels show significant voxels that discriminate between the emotional states. In the present application, however, FIG. 9 is presented in gray scale for printing clarity purposes. The intensity of the gray corresponds to the various colors in the typical GLM and SVM maps.

FIG. 10: OPERATIONAL FLOWS REPRESENTING ILLUSTRATIVE EMBODIMENTS OF OPERATIONS RELATED TO DETERMINING BRAIN STATES.

FIG. 11: Operational flows representing illustrative embodiments of operations related to determining brain states.

FIG. 12: Diagram of an illustrative embodiment of a system useful for determining brain states.

FIG. 13: Operational flows representing illustrative embodiments of operations related to determining brain states.

FIG. 14: Illustrative embodiment showing a partial view of an embodiment of the operational flow of FIG. 13.

FIG. 15: Operational flows representing illustrative embodiments of operations related to determining emotional states.

FIG. 16: Illustrative embodiment showing a partial view of an embodiment of the operational flow of FIG. 15.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Described herein are multivariate, pattern-based apparatus, systems and methods for recognition of brain states from spectroscopic data and generation of a read-out of both the spatial and temporal patterns. The system can be applied, for example, for enhancing a desired function or behavior, or to alleviate a behavioral or neurological problem. The system can also be used to investigate the spatiotemporal evolution of a network of brain activity corresponding to a brain function, by changing the activity as an independent variable and studying its effect on behavior.

Cognitive, perceptual, motor and emotional states differ in both their spatial and temporal signature, in terms of time evolution of activity in one region and also in terms of dynamic interaction with other regions of the brain. Systems and pattern-based methods that incorporate both spatial and temporal information in the analysis use considerably more information for detecting the current “mental” state from measurements of brain activity. Access to this analysis online and in real-time is of great practical use for BCIs, neurofeedback systems for clinical applications, neurophysiologic treatment and neuroscience research.

As used herein, the phrase “Brain-Computer-Interfaces” or “BCI” is an interface that enables control of computers and of external devices using brain activity regulation. BCI refers to a signal-processing circuit that enables the control of computers and other external devices using brain activity regulation. The BCI may be invasive such as with implanted electrodes, and/or noninvasive, such as with electroencephalography and MRI.

As used herein, the phrase “brain-computer-interface system” and “BCI system” refer to multiple components including a BCI as defined above, a means of generating raw brain signals such as, but not limited to, EEG, MRI, and/or NIRS, computer(s) or computing capability and optionally other devices, that together serve the function of translating raw brain signals which then may optionally be sent to an output of a device,

As used herein, “BOLD” stands for blood-oxygen-level dependent. Activity due to stimulation in a tissue generates a temporary lack of oxygen in the blood surrounding the tissue. Once this lack of oxygen is detected, additional oxygen is supplied to the tissue where the automatic controller action providing the oxygen supply generally overshoots the original oxygen content. Thus, a long-term-decay of the increased oxygen content back to baseline oxygen levels can be observed. This chronological modification of the local oxygen content in the blood is measured and localized in BOLD-fMRI experiments using a nuclear magnetic resonance tomography apparatus.

As used herein, the term “magnetic resonance imaging”, or “MRI” is a spectroscopic method using perturbations of magnetic nuclei in a magnetic field. MRI allows for non-invasive recording of neuronal activity across the entire brain with relatively high spatial resolution and moderate temporal resolution (in the range of millimeters and seconds, respectively). MRI enables whole brain coverage, and extraction of signals from cortical and subcortical regions of the brain with high anatomical specificity. MRI has been used successfully to study blood flow in vivo. U.S. Pat. Nos. 4,983,917, 4,993,414, 5,195,524, 5,243,283, 5,281,916, 5,184,074 and 5,227,725. Such techniques are generally directed to measuring the signal from moving moieties (e.g., the signal from arterial blood water) in the vascular compartment, and image water flowing in the arteries.

As used herein, the term “functional magnetic resonance imaging”, or “fMRI” refers to an MRI technique which non-invasively measures the task induced blood oxygen level-dependent (BOLD) changes correlating with neuronal activity in the brain.

As used herein, the term “diffusion MRI” refers to an MRI technique which non-invasively measures local microstructural characteristics of water diffusion. Image-intensities at each point are determined by the strength and direction of the magnetic diffusion gradient, as well as on the local microstructure in which the water molecules diffuse.

As used herein, the term “Near Infrared Spectroscopy” or “NIRS” means the spectroscopic method using the near infra-red region of the electromagnetic spectrum (from about 800 nm to 2500 nm). NIRS can be used for non-invasive assessment of the brain function using, for example, BOLD. This technique is also known as optical topography (OT), and is used interchangeably herein. A Near Infrared Spectrascope is an optical imaging device using NIRS.

As used herein, the term “univariate analysis” is one of many analysis methods that analyses a signal, such as BOLD signal from multiple (often many thousands) of locations repeatedly, but then analyzing each location separately.

As used herein, the term “real time” means within a time interval useful for learning to control the feedback and thus control brain activity or observing and reacting to the readout. Thus, the term real time is in relation to the subject brain response, not data flow, and may be measured in seconds, or milliseconds. In some embodiments, real time processing occurs within approximately 5 seconds. In some embodiments, real time processing occurs within approximately 2 seconds. In other embodiments, real time processing occurs within approximately 1.5 seconds. In other embodiments, real time processing occurs within approximately 1 second. In some embodiments, real time processing occurs within approximately 500 milliseconds. Thus, in some embodiments, the real time processing occurs within approximately 1 millisecond to 5 seconds. In another embodiment, the real time processing occurs within approximately 1 millisecond to 3 seconds. In another embodiment, the real time processing occurs within approximately 1 millisecond to 1 second. In another embodiment, the real time processing occurs within approximately 1 millisecond to 500 milliseconds.

As used herein, the term “subject” refers to any mammalian organism, which will undergo an MRI scan or other spectroscopic imaging and is capable of responding to feedback from the system as described herein. In one embodiment, the subject is a human. In another embodiment, the subject is a mouse, rat, monkey, pig, dog, cat, horse, rabbit, dolphin, whale, shark, or other animal. In some embodiments, the subject is a domestic animal, a wild animal, a zoo, aquarium, exhibition, or show animal, an animal used for racing, a working animal (e.g. with the blind, or in forensics, etc.), a laboratory animal, etc.

As used herein, the term “pattern-based multivariate analysis” is a multivariate, pattern-based method that can, for example, use advanced machine learning techniques, such as multilayer neural networks, support vector machines and so forth to discriminate spatial, temporal and spectral patterns. Pattern-based multivariate analysis has several advantages over conventional univariate analysis including but not limited to: (1) weak information available from single locations can be accumulated across many spatial locations; (2) interaction between brain regions can be determined by simultaneously analyzing activity in multiple locations; (3) temporal evolution of activity in different regions and their interactions can be different for different brain functions; and (4) conventional neuroimaging uses preprocessing methods such as spatial smoothing, which may remove important information about the brain state.

As used herein, the term “regions of interest” or “ROI” are regions in the brain that have been selected as a region having enhanced activity compared to other regions.

As used herein, the term “selecting” means any process used to identify for use one or more target components. Processes include, but are not limited to, user selected, user identified, software method analysis, algorithm-based, computer mediated, operations research, optimization, simulation, queuing theory, and/or game theory.

One embodiment is an apparatus which comprises a real-time spatiotemporal pattern classifier configured to classify spatial and/or temporal spectroscopic data of brain states in real time, where the classifier includes a feature extraction module and a pattern classification module; and a read-out having spatial and/or temporal components. In one embodiment, the real-time spatiotemporal pattern classifier is configured to classify both spatial and temporal spectroscopic data and the read-out has both spatial and temporal components.

In some embodiments, the apparatus comprises a real-time spatiotemporal pattern classifier configured to classify spatial and/or temporal data such as MRI or optical imaging data of brain states in real time.

In some embodiments, the feature extraction module comprises a data driven algorithm or a model driven algorithm. In other embodiments, the feature extraction module comprise a principal component analysis (PCA) model. In another embodiment, the feature extraction module comprises both a data driven algorithm and a model driven algorithm.

In some embodiments, the apparatus further comprises a signal preprocessor. Optionally, the signal preprocessor processes the signal prior to classification.

In some embodiments, data driven algorithm comprises a principal component analysis (PCA) model. In other embodiments, the data driven algorithm comprises an independent component analysis (ICA) model. In yet other embodiments, the data driven algorithm comprises a general linear model (GLM) and a Granger causality model (GCM). In yet other embodiments the data driven algorithm comprises both a principal component analysis (PCA) model and an independent component analysis (ICA) model.

In some embodiments, the model driven algorithm is configured to extract features from a region of interest selected by examining spatial patterns based on a network connectivity of a brain activity. In other embodiments, the model driven algorithm comprises a general linear model (GLM).

In some embodiments, the pattern classification module comprises a Hidden Markov Model (HMM) or a Support Vector Machine (SVM).

In some embodiments, the read-out is a signal feedback device. In some of these embodiments, the read-out device comprises one or more graphical meters or iconic representations.

In some embodiment, the classifier is configured to classify the data in less than 5 seconds, or less than 4 seconds, or less than 3 seconds, or less than 2 seconds, or less than 1 second, or less than 800 milliseconds, or less than 500 milliseconds. In some embodiments, the real-time spatiotemporal pattern classifier is configured to classify data in about 0.5-5.0 seconds. In one embodiment, the data is classified in about 0.5-3.0 seconds. In another embodiment, the data is classified in about 0.5-2.0 seconds. In another embodiment the data is classified in about 0.5-1.0 seconds. In another embodiment, the data is classified in about 100 milliseconds to 1.0 second.

In some embodiments, the feature extraction module and the pattern classification module comprise at least two separate modules. In other embodiments, the feature extraction module and the pattern classification module are a single module.

One embodiment is a system which comprises a spectroscope; a real-time spatiotemporal pattern classifier configured to classify spatial or temporal data in real time, which includes a feature extraction module and a pattern classification module; and a read-out having spatial and/or temporal components.

In some embodiments, the system is a brain-computer-interface system.

In some embodiments, the real-time spatiotemporal pattern classifier is configured to classify both spatial and temporal data and the read-out has both spatial and temporal components.

In some embodiments, the system further comprises a signal preprocessor.

In some embodiments, the system is configured to measure a brain state in a subject.

In some embodiments, the spectroscope is a magnetic resonance imaging (MRI) device. The MRI device may be, for example a functional MRI device (fMRI), where the fMRI may be configured to utilize an echo planer imaging sequence. In other embodiments, spectroscope is a near infrared spectroscope (NIRS).

Another aspect comprises the method of obtaining a spectroscopic signal from a subject brain, the signal having spatial or temporal data; extracting one or more features from the signal in real time; classifying a pattern of a brain state in real time; and generating a read-out comprising the spatial and/or temporal data.

In some embodiments, the real-time spatiotemporal pattern classifier is configured to classify both spatial and temporal data and the read-out comprises both spatial and temporal components.

In some embodiments, two or more brain states are classified in real time.

In some embodiments, the read-out is associated with a specific physical, cognitive, or emotional task of the subject.

In some embodiments, the read-out is configured to provide feedback to the subject. In other embodiments, a person (e.g. researcher, laboratory technician, nurse practitioner, physician, etc.) observes and optionally acts on the read-out.

In some embodiments, the method also comprises enhancing a desired function or behavior in the subject or alleviating a behavioral or neurological problem in the subject by using the feedback to affect the subject's brain state.

In some embodiments, the method also comprises recognizing a brain state in the subject brain.

In some embodiments, the method also comprises recognizing, diagnosing, or treating a disease or condition in the subject. In some embodiments, the condition is the utterance of a false statement. In other embodiments, the condition is reduced limb function. In other embodiments, the condition is addiction. In yet other embodiments, the disease is a neurological disorder or condition, psychopathy, phobia, anxiety, depression or schizophrenia.

Yet another embodiment is a device where the device comprises: a means for obtaining a spectroscopic signal from a subject, wherein the signal includes spatial or temporal components; a means for real-time classification of the spectroscopic signal and a means for obtaining a read-out comprising spatial and/or temporal components.

In one embodiment, the real-time spatiotemporal pattern classifier is configured to classify both spatial and temporal data and the read-out to include both spatial and temporal components.

In one embodiment, the device includes a means for processing the spectroscopic signal.

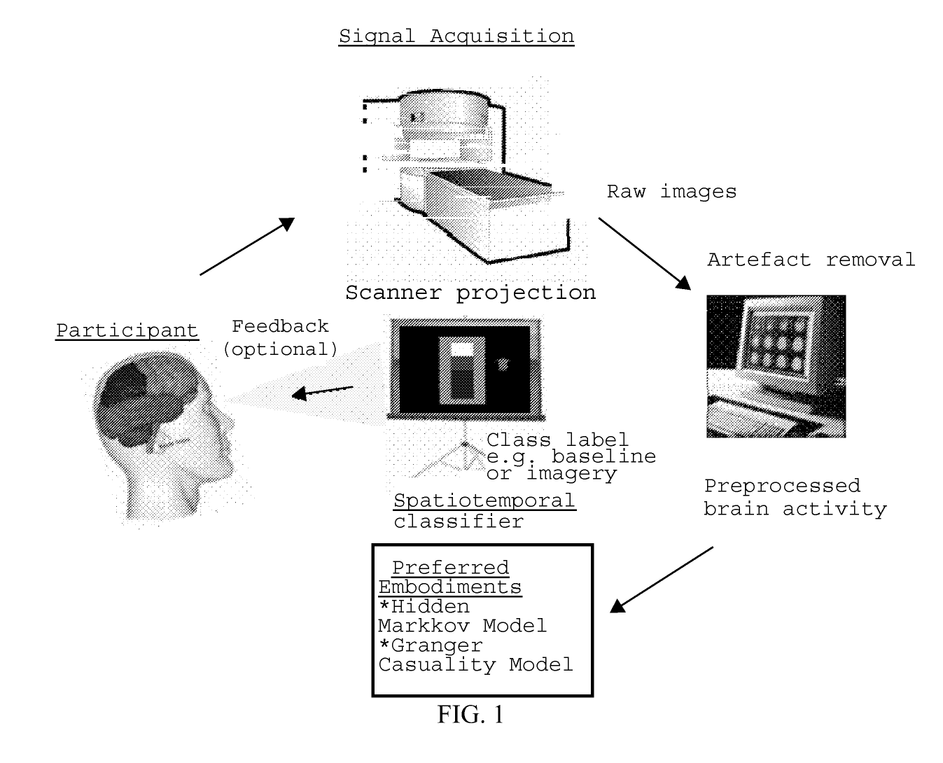

One embodiment comprises a fMRI-BCI system which is a closed loop system (FIG. 1), with the following major subsystems: signal acquisition, signal preprocessing, spatiotemporal pattern classification and signal feedback. The subsystems may be installed and executed in separate computers for optimising system performance, and may be connected via a local area network (LAN).

Another embodiment comprises a fMRI system with the following major subsystems: signal acquisition, signal preprocessing, spatiotemporal pattern classification and signal read-out.

Another embodiment comprises a fMRI-BCI system with the following major subsystems: signal acquisition, spatiotemporal pattern classification and signal read-out.

Another embodiment comprises a NIR system with the following major subsystems: signal acquisition, signal preprocessing, spatiotemporal pattern classification and signal read-out.

The apparatus and system described herein can be used for a variety of different applications, some of which are discussed herein below. An embodiment provides for the automatic detection and recognition of motor, sensory, cognitive and emotional states of the brain using spectroscopic signals such as, but not limited to, functional magnetic resonance imaging (fMRI) signals, online and in real-time using multivariate pattern-based analysis. This and the associated method of using the system allow for multiple uses in recognizing, diagnosing or treating a disease or condition in a subject. The system and associated methods are also useful in the study of the brain and brain function, including emotional content.

While other methods of neuroimaging, such as EEG and MEG-based classification of brain states provide images of the brain, as described herein the classification extracts and uses signals from localized as well as spatially distributed regions of the brain, including both cortical and subcortical regions of the brain. As a result, brain states of emotion, cognition and perception can be discriminated from deeper brain areas, such as insula, cingulated cortex, thalamus, hippocampus, brain stein and even cerebellum. Discrimination of complex cognitive, emotional and perceptual states is possible with the systems and methods described herein. Thus, an embodiment includes discrimination, study, and diagnosis of such emotional and perceptual states.

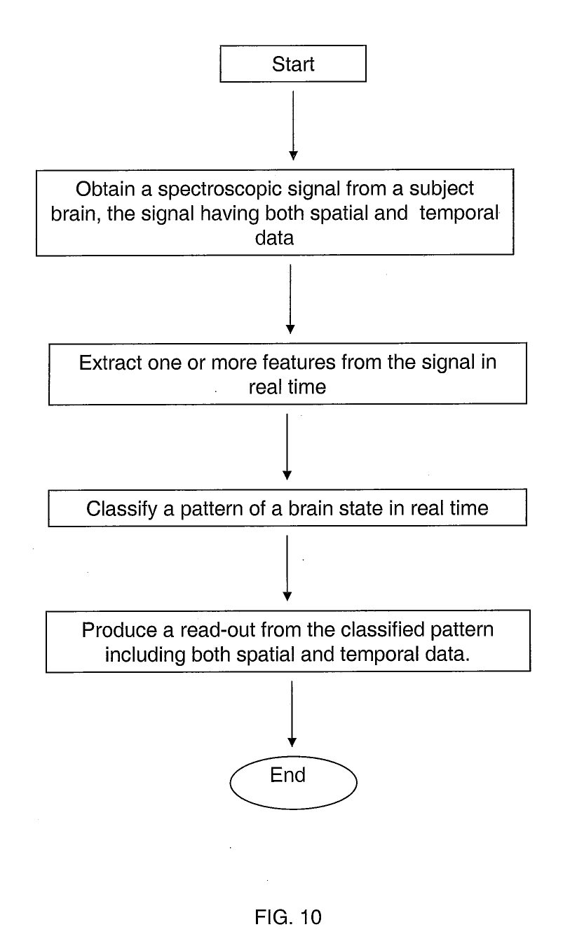

FIG. 10 shows an operation flow representing an illustrative embodiment of the operation of the method for determining brain states. After a spectroscopic signal, containing both spatial and temporal data has been obtained from a subject brain, the operational flow moves to an operation of extracting one or more features from the signal in real time using a feature extraction module. Examples include, but are not limited to, the data driven algorithm, principal component analysis (PCA), and the model driven algorithm, general linear model (GLM). The operational flow then moves to classifying a pattern of brain state in real time. The operational flow then moves to producing a read-out from the classified pattern including both spatial and temporal data. The operational flows may also be executed in a variety of other contexts and environments, and or in modified versions of those described herein. In addition, although some of the operational flows are presented in sequence, the various operations may be performed in various repetitions, concurrently, and/or in other orders than those that are illustrated.

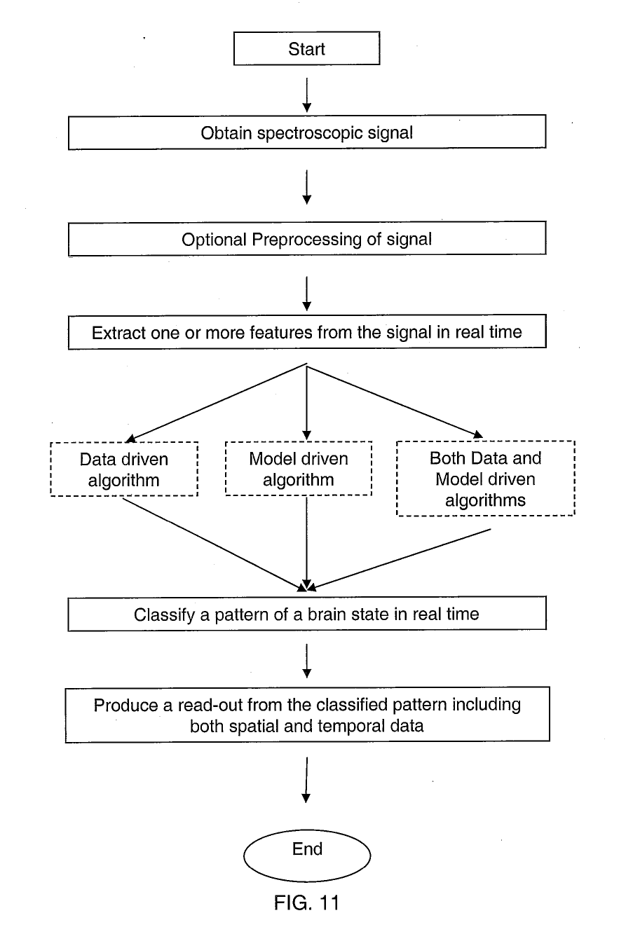

FIG. 11 shows an operational flow representing an illustrative embodiment of the operation of the method for determining brain states. A spectroscopic signal is obtained. Optionally, the operational flow moves to preprocessing the signal. The operational flow then moves to extracting one or more features from the signal in real time using a feature extraction module. This feature extraction module is optionally a data driven algorithm, a model driven algorithm, or both a data and a model driven algorithm. The operational flow then moves to classifying a pattern of brain state in real time. The operational flow then moves to producing a read-out from the classified pattern including both spatial and temporal data.

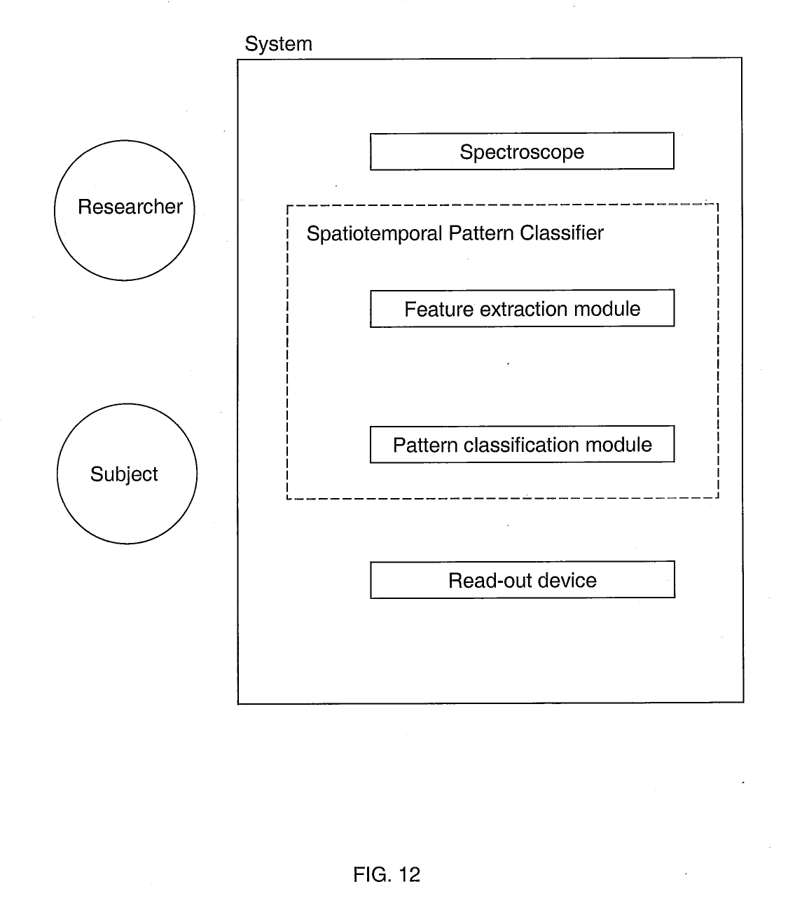

FIG. 12 shows an illustrative embodiment of a system. The system includes a spectroscope, and a spatiotemporal pattern classifier configured to classify both spatial and temporal data in real time. The spatiotemporal pattern classifier includes, but is not limited to, a feature extraction module and a pattern classification module. The system also optionally includes a read-out device. The read-out device may be read by one or more of a researcher, physician, nurse practitioner, laboratory technician, or subject. Optionally, another person or persons may read the read-out device. This is particularly useful in an embodiment wherein the subject's response and/or the images, instructions, etc presented to the subject are recorded and the other person or persons are simultaneously presented the read-out device and the subject's response and/or the images, instructions, etc.

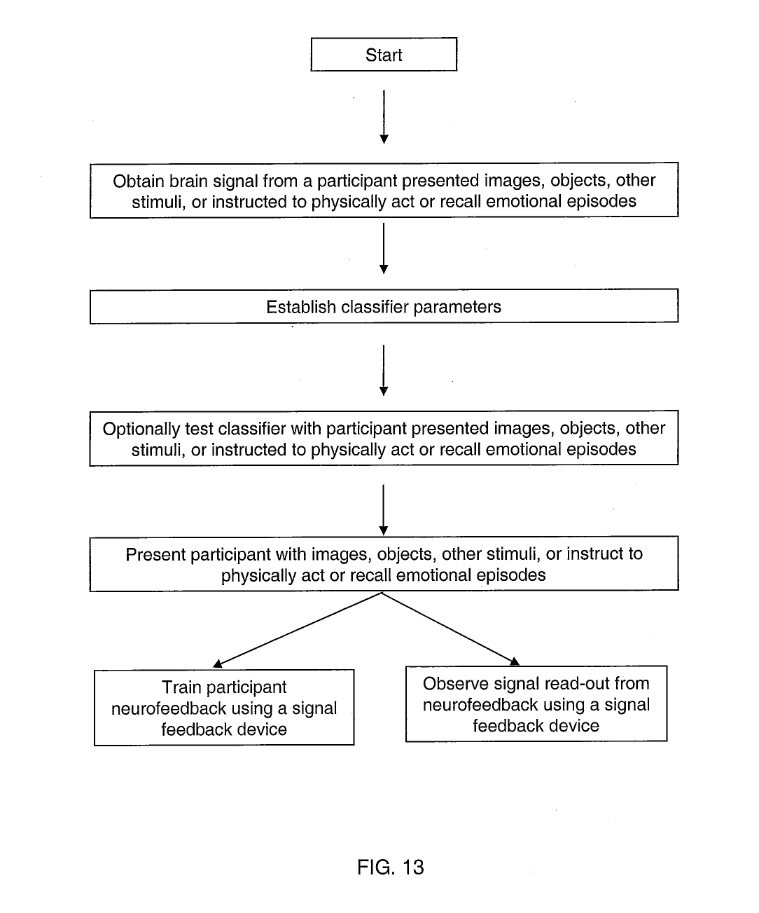

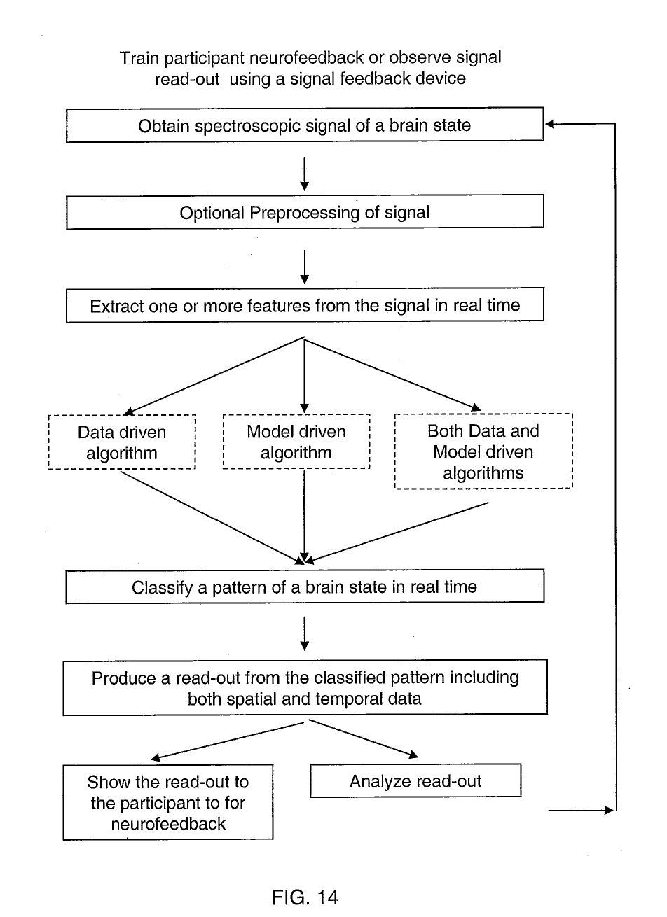

FIG. 13 shows an operation flow representing an illustrative embodiment of the operation of the method for determining brain states. A brain signal is obtained from a participant presented images, objects, other stimuli, or instructed to physically act or recall emotional episodes. The operational flow then moves to establishing the classifier parameters. The operational flow then moves to optionally test classifier with participant presented images, objects, other stimuli, or instructed to physically act or recall emotional episodes. Next, the operational flow moves to presenting the participant with images, objects, other stimuli, or providing instructions to physically act or recall emotional episodes. The operational flow then moves to either training participant neurofeedback using a signal feedback device or to observing signal read-out from neurofeedback using a signal feedback device as described in FIG. 14 which shows an operational flow representing an illustrative embodiment of the operation of the method for determining brain states. Initially, as exemplified in FIG. 14, a spectroscopic signal is obtained. Optionally, the operational flow moves to preprocessing the signal. The operational flow then moves to extracting one or more features from the signal in real time using a feature extraction module. This feature extraction module is a data driven algorithm, a model driven algorithm, or both a data and a model driven algorithm. The operational flow then moves to classifying a pattern of brain state in real time. The operational flow then moves to producing a read-out from the classified pattern including both spatial and temporal data.

In one embodiment, after a spectroscopic signal containing both spatial and temporal data has been obtained from a subject brain the operational flow moves to an operation of extracting one or more features from the signal in real time using a feature extraction module. An example of this is the data driven algorithm, principal component analysis (PCA), and the model driven algorithm, general linear model (GLM). The operational flow then moves to classifying a pattern of brain state in real time. The operational flow then moves to producing a read-out from the classified pattern including both spatial and temporal data. The operational flows may also be executed in a variety of other contexts and environments, and or in modified versions of those described herein. In addition, although some of the operational flows are presented in sequence, the various operations may be performed in various repetitions, concurrently, and/or in other orders than those that are illustrated.

In another embodiment, the system comprises a spectroscope which produces data having both spatial and temporal characteristics, wherein the spectroscopic data is provided to an optional preprocessor. The data is then provided to a real-time spatiotemporal pattern classifier configured to classify both spatial and temporal spectroscopic data of brain states in real time, which includes a feature extraction module and a pattern classification module. The feature extraction module comprises a data driven algorithm, a model driven algorithm, or both a data driven algorithm and a model driven algorithm. Exemplary data driven algorithms include principal component analysis (PCA) model, independent component analysis (ICA) model, and general linear model (GLM) and a Granger causality model (GCM). Exemplary model driven algorithms include the general linear model (GLM). The model driven algorithm may be configured to extract features from a region of interest selected by examining spatial patterns based on a network connectivity of a brain activity. The pattern classification module may comprise a Hidden Markov Model (HMM), a support vector machine (SVM), or other known pattern classification modules. The classified data is then sent to a read-out device having both spatial and temporal components. This read-out device may be a signal feedback device such as a graphical meter or iconic representation. Readout may be simply an output signal. Optionally, this signal can be read on any device which is proximate to the subject or at any other location. Such devices may include for example, monitors, sensors, mobile phones, computers, etc. The readout may be sent to the readout device via wired or wireless communication.

In one embodiment, the apparatus, systems, and methods described herein may be used as a neuroimaging tool. For example, the emotional state of a patient may be determined using the methods as described herein. This can be done automatically, i.e., by a computer or machine with minimal or no human intervention once the system has been set up, and it can be done in real-time (i.e., milliseconds or seconds after the acquisition of the brain signals). This method may be used in brain-computer interfaces (BCIs), neurofeedback systems, clinical treatment of mental disorders including but not restricted to depression, schizophrenia, anxiety, psychopathic and social phobia, automatic detection of deception in criminals and persons endangering security, development of affective and socially competent computers, intelligent machines and robots that recognize and express emotions.

In one embodiment, the apparatus, systems, and methods described herein can be used in behavioral therapy to provide real-time feedback to a patient needing behavioral treatments. This is also useful in the field of psychiatric treatment, and may similarly be used to train the patient to modify one or more brain state to treat a psychiatric aliment.

Particular fields where embodiments as described herein may be used include neuroscience, neuroradiology, medical psychology, machine learning, signal processing and affective computing.

Spectroscopic signal acquisition is performed by a spectroscope which is an instrument that measures the interaction between radiation and matter as a function of wavelength or frequency. Spectroscopic signals include MRI and NMR, IR, NIR, Raman, Absorbance, UV Fluorescence, Xray, Photoemission, and Mossbauer. The spectroscope as useful herein is able to provide spectroscopic signals which provide information on neural activity and thus brain states.

In one embodiment, magnetic resonance imaging (MRI) is used. The MRI signal acquisition is accomplished by any MRI technique known to one of ordinary skill in the art.

In one embodiment, the MRI technique is functional MRI (fMRI). In one embodiment, the fMRI provides whole brain images from healthy subjects are acquired employing a conventional echo planar imaging (EPI) sequence or any of its variants. The measured hemodynamic response due to the BOLD effect, which is the neurovascular response to brain activity, lags behind the neuronal activity by approximately 3-6 s. Higher static magnetic field (B0) strengths and more sophisticated MRI pulse sequences can be used to increase the signal-to-noise ratio (SNR).

In another embodiment, the MRI technique is diffusion MRI, which measures local microstructural characteristics of water diffusion. Image-intensities at each point are determined by the strength and direction of the magnetic diffusion gradient, as well as on the local microstructure in which the water molecules diffuse.

For diffusion-weighted imaging (DWI, or dwi MRI), three gradient-directions are applied to provide the average diffusivity. For diffusion tensor imaging (DTI) scans comprise at least six gradient directions are applied to compute the diffusion tensor. Additional diffusion MRI methods that may be used include q-space imaging and generalized diffusion tensor imaging.

Images are acquired in a given paradigm depending on the application for which the data is sought. Some examples of MRI scanners useful are 3T Siemens TIM Trio Scanner, Siemens Medical Systems, and Erlangen. In one embodiment, an echo planar imaging (EPI) sequence is used.

In another embodiment, the imaging device is an optical imaging device, such as Near Infrared Spectroscope (NIRS). NIRS can be used for non-invasive assessment of the brain function using BOLD. This technique is also known as optical topography (OT), and is used interchangeably herein.

The spectroscopic data optionally pre-processed using one or more of a variety of methods. Preferably, the preprocessing occurs in real-time. A single pre-processing step may be used, or two or more different steps may be performed on the data in any order. In a representative embodiment, thee acquired images are preprocessed to correct for head motion and to compensate for signal dropouts and magnetic field distortions.

A non-limiting list of the preprocessing steps which may be used includes: spatial smoothing; temporal filtering; slice time correction; transformation into standard coordinates; resampling of data; motion correction of data; regression filtering; and selection of voxels corresponding to the brain. Preprocessing may be performed prior to the classification. Alternatively, the preprocessing may be done at any point in the process.

Spatial smoothing may be accomplished according to standard methods to produce smoothed output data. This is useful because it removes noise in the data, improves statistical properties by making the data variance more Gaussian, and produces an image that is easier to interpret visually. This may be accomplished by convolving the data with a 2-D or 3-D Gaussian filter function with a defined half-width, among other methods.

Temporal filtering includes lowpass, highpass, and bandpass filtering and convolving with a function such as a hemodynamic response function. This is useful because it removes temporal noise in the data, matches the signal power in the data to that corresponding to the trials being conducted, and improves later data processing and statistical measures. This is accomplished by convolving the data with a temporal filter. This convolution will normally be with a causal filter as the data is being collected in substantially real time. The filter can be a highpass filter, such as a highpass filter with the cutoff of, for example, 10, 30, 60, 120, 240, or 300 s, or the lowest relevant frequency component of the behavioral trials being conducted, or a drift rate that reflects the slowest relevant physiological change expected in the signal. The filter can be a lowpass filter, such as a lowpass filter or Gaussian function with the cutoff of, for example, 0.10, 0.25, 0.5, or 1 s. The filter can be a lowpass filter designed to match the shape of a hemodynamic response function modeled as an alpha function. The filter can be a bandpass filter that accommodates a combination of highpass and lowpass characteristics. These filters can be designed using standard digital filter design techniques.

Slice time correction to correct for the time of collection of each slice by interpolation may be used. This is useful because it approximates the case where each slice in a scan volume is collected simultaneously. In order to perform this computation, the relative times of collection for each slice in a scan volume are known. The first image in each volume is taken as the reference image. The output values for each successive image in the volume are computed as the interpolated value between the measured value for each voxel in the image and the measured value for the same voxel in the previous image or succeeding. The interpolation yields the value corresponding to the estimated value for the voxel at the time point actually measured for the reference image.

Transformation into standard coordinates may be accomplished by applying a transformation vector that yields the corresponding value at each voxel in a standard coordinate space. This has the advantage that all subsequent processing and display of data is in a standard coordinate space such as Talairach space or MNI space that can be directly compared with reference data.

Resampling may be accomplished to increase or decrease the temporal and spatial resolution of the data, using band-limited filtering if needed. Resampling can produce a more detailed or less detailed view of the collected data and can be performed using standard methods.

Motion correction may be accomplished to adjust for the motion that takes place between subsequent scans. This is useful because each section of each volume is in substantially the same position as in the first or reference scan of a scanning session. This can take place by applying using a transform created for each scan volume to that scan volume. The transform is designed to create the best fit in the least-squared error sense between the data of the current scan and the reference scan, including translation, rotation, and scaling if needed. An example of this software is described in: C C Lee, et al. Magn Reson Med 1996; 36:536-444. In one embodiment, ICA is used to correct for subject movement.

Regression filtering can be used to remove noise components associated with exogenous events. For example, the activity level in each voxel may be correlated with an event not directly related to testing, such as, but not limited to, the phase of the cardiac or respiratory cycle, or movement of the subject brain. The data from each voxel may be corrected by regressing out this noise source. This method is described in the literature, for example in J. T. Voyvodic, Neurolmage 10, 91-106 (1999).

In one embodiment, the selection of voxels corresponds to the region of interest in the brain. This process may include the masking off of voxels determined to be outside of the region corresponding to the brain, such as voxels corresponding to the skull and regions outside of the head. This process may also include the masking on of voxels determined to be inside the region corresponding to the brain. This process may take place automatically under software control. Algorithms for this process are described in the literature and are known to one skilled in the art.

Spatiotemporal classification involves classifying brain states using the distinct pattern of activity in space (across different voxels of the brain) and time (across different time points of brain state or the task). A spatiotemporal pattern classifier as described herein classifies by: 1) Feature Extraction and 2) Pattern Classification.

Spatiotemporal classification that occurs in real time is provided herewith. In contrast to the conventional method of offline analysis occurring long after the brain (or other) scan is completed and the subject is removed from the scanner, the real-time nature of the methods, devices, and apparatus as described herein allows for online investigation of the functional activation of the brain, in applications such as clinical treatment of neurological disorders, surgical preparation, and incorporation of interactive teaching methods for students of neuroscience.

Feature extraction is performed by a feature extraction module to reduce the input dimension and to extract useful information for the classifier and is generally used to process large data input. Feature extraction transforms the input data into the set of features and include algorithms and methods that reduce the dimensionality of the data.

A limitation of the prior implementations of feature extraction in real-time classification of spectroscopic signals is generally their over-reliance on data driven methods (i.e., using BOLD-intensity thresholds or Analysis of Variance (ANOVA) for fMRI analysis). Such methods tend to ignore the existing knowledge and understanding of the brain activations during action, perception, cognition and emotion. Embodiments as described herein incorporate: 1) data driven methods such as Principal Component Analysis (PCA) and Independent Component Analysis (ICA), 2) model-driven methods for feature extraction, or 3) a combination of data and model-driven methods.

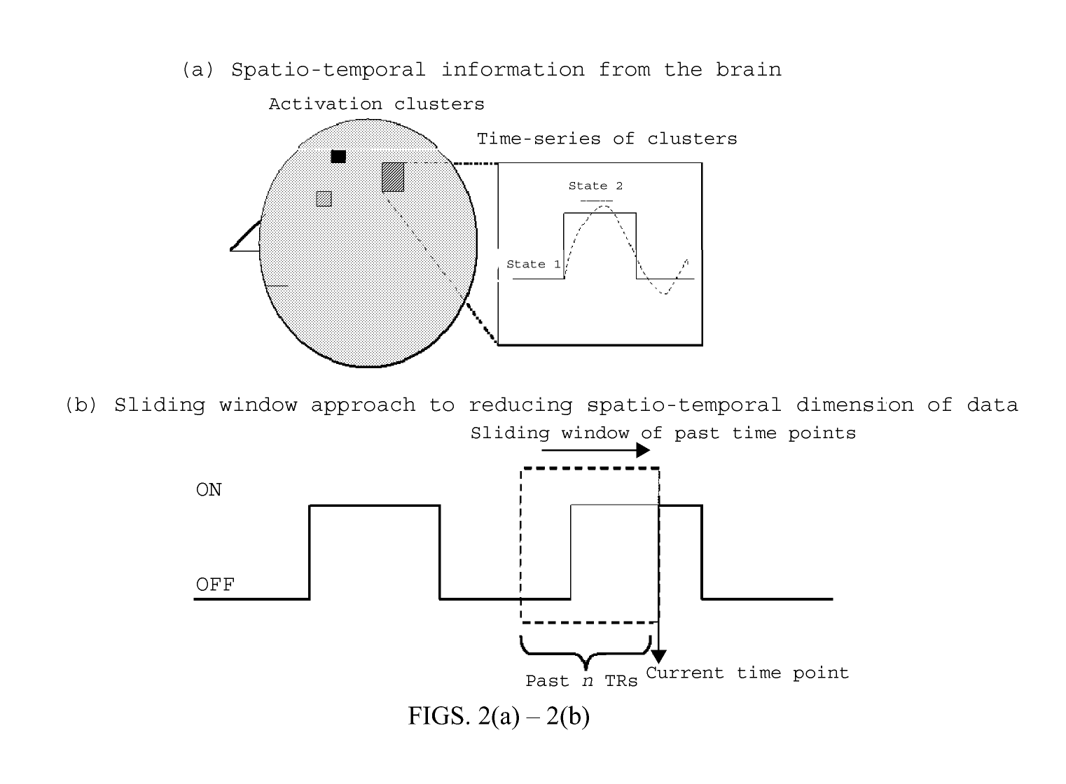

The system is implemented such that both data driven and model driven approaches of feature extraction are made available as options that can be selected by the scientist/clinician in either a set or an exploratory way. The selection of which method or to combine both methods is dependent on the particular data. In particular, if there is a wealth of knowledge from prior application of embodiments as described herein or from data available to a person of ordinary skill in the art regarding which areas of the brain will be active for a particular spectroscopic signal, this information leads to the use of the model driven method. One or both methods can also be employed one after another to improve performance of classification. FIG. 2 shows the use of both spatial and temporal information (from a sliding window of past n time points) from regions of the brain for pattern classification.

The data driven algorithms are performed on the data useful in the embodiments, and are algorithms that can provide real-time data read-out where the algorithm parameters are based on data. Feature extraction transforms the input data into the set of features and includes general dimensionality reduction algorithms such as: principal component analysis (PCA, including kernel PCA and also called Karhunen-Loève transform-KLT), independent component analysis (ICA), general linear model (GLM), Granger causality model (GCM), semidefinite embedding (SDE), multifactor dimensionality reduction (MDR), nonlinear dimensionality reduction (NDR), isomaping, latent semantic analysis (LSA), partial least squares (PLS), and Fisher Criterion (FC). Other data driven algorithms may also be used.

Principal Component Analysis (PCA) and Independent Component Analysis (ICA) have several generic features in common. For example, both PCA and ICA include a reduction of data dimension in the spatial dimension so that temporal and spectral aspects of the fMRI data can be combined with the spatial components for pattern classification. Further, both can be used to extract useful features from spatial, temporal and spectral dimensions of the fMRI data for pattern classification. Additionally, both PCA and ICA can be used for developing an automatic pattern classification system with minimal human knowledge, expertise and effort. Further both PCA and ICA are able to perform pattern classification with minimal computing and time resources. PCI, and ICA, however, have several differences as follows.

### Principal Component Analysis (PCA)

PCA is a method for compressing a set of high dimensional vectors into a set of low dimensional vectors and then reconstructing the original set. It is a non-parametric analysis, and hence the end result is the same and independent of any hypothesis or assumption about data probability distribution. PCA operates on the following assumptions: (1) linearity, (2) statistical importance of mean and covariance, and (3) that large variances have important dynamics. The assumption on linearity means that the observed data set is assumed to be linear combinations of certain basis. Non-linear methods, however, exist such as kernel PCA which do not assume linearity. The assumption on the statistical importance of mean and covariance states that there is no guarantee that the directions of maximum variance will contain good features for discrimination. When PCA is used for clustering, its main limitation is that it does not account for class separability since it makes no use of the class label of the feature vector. The assumption that large variances have important dynamics implies that PCA simply performs a coordinate rotation that aligns the transformed axes with the directions of maximum variance. When the observed data has a high signal-to-noise ratio, the application of PCA may result in interesting dynamics and lower noise.

### Independent Component Analysis (ICA)

ICA is used when the assumption that the signal sources are independent is correct. The use of the blind ICA method of mixed signals gives very good results. Further, ICA is used in investigating signals that are not generated by mixing as artefacts or by-product of analysis steps. The application of ICA is made simpler by assuming there are no time delays and/or echoes. ICA is a statistical method for finding independent components (e.g., factors, latent variables and sources) by maximizing the statistical independence of estimated components. Independence of components may be measured by: (a) Non-Gaussianity, as measured by negative entropy or (b) kurtosis Mutual information.

Principal Component Analysis (PCA) is a technique used to reduce multidimensional data to lower dimensions for analysis. PCA usually involves the mean centering of the data and then the application of Singular Value Decomposition. PCA transforms mathematically the data to a new coordinate system such that the greatest variance by any projection of the data would lie on the first coordinate called the first principal coordinate, the second greatest variance on the second coordinate, and so on. In terms of least squares, PCA is the optimum transform for a given data set. PCA can be used for dimensionality reduction in a data set, by retaining those characteristics of the data set that contribute to its most variance—that is by keeping lower order principal components and ignoring the higher order ones.

In one embodiment for a fMRI data set X^{T}, after computing zero mean and subtracting that from the original data, a matrix of data is formed such that each row represents the BOLD intensities at each voxel in the brain and each column represents the time points, from the current time point to the last n points, the PCA transformation is given by:

Y^{T}=X^{T}W=VΣ
where VΣW^{T} is the Singular Value Decomposition (SVD) of X^{T}.

PCA, however, is limited since no a priori model assumptions can be incorporated. In addition, the PCA often incurs loss of information. Thus, PCA us used in embodiments where the direction of maximum variance as defined by the PCA model contains acceptable features for discrimination in a pattern classifier (i.e., changes in BOLD intensities and not micro head movements provide for maximum variance).

In order to overcome the above limitations, as necessary, Independent Component Analysis and model-based feature extraction as described herein below can be used. Nevertheless, PCA is a non-parametric analysis that gives a unique answer for compression of a set of high-dimensional vectors to lower dimension and may be applicable to certain systems and methods as described herein and in particular for exploring improvement in brain state recognition.

Real-time Independent Component Analysis (ICA) decomposes the acquired time-series of the voxels into a set of spatial maps or components and their associated time-courses. Two variants of ICA, namely, spatial and temporal could be separately applied to generate spatial and temporal components, respectively. Spatial ICA models the spatial distribution of brain activity and builds statistically independent maps or components, while the temporal ICA generates components with different time correlations. ICA has largely been used as an offline analysis method for fMRI data. However, recently real-time implementations of ICA have been developed (Esposito, F., et al., Neuroimage. 20, 2209-2224, 2003). In one embodiment, an adaptation of the real-time ICA for the purpose of real-time feature extraction for online pattern classification of brain states is incorporated.

The real-time application of ICA is based on the configuration of several important parameters of input data vectors. These configurations are related to: the length of a temporal window from which the ICA is computed (denoted by L), repetition time of the scans (D), and the frequency of update of the components (S). The procedure for the algorithm is as follows. Once the temporal window is filled with new data, the ICA computation will be launched. Any of the different ICA algorithms developed so far could be potentially applied for this purpose. One algorithm is the Fixed Point Algorithm by (Hyvarinen and Pajunen, Neural Netw. 12, 429-439, 1999).

Within the Fixed Point Algorithm, the temporal dimension T of the dynamic computation of ICA equals the number of scans included in the window as:

T=Integer(L/D).

After S newly acquired scans, a varying number of IC maps are generated, ranging from zero (when no convergence is achieved within the maximum number of iterations) to a maximum number of T independent components.

Two important choices are made: (1) the maximum number of ICs to be generated and (2) the number of ICs to be used in the feature extraction process. The first choice relates to the trade-off between the computational load (dependent on L,D,S and the TR) and the number of distinct activation patterns that is desired for successful classification. The second choice relates to the reduction of input dimension to the classifier while maintaining high accuracy of classification.

When the independent components are generated, two different methods could be used to select them as input for the classifier: (1) selection based on temporal correlation with the experimental protocol, (2) selection based on overlap with a spatial mask of the brain regions. The former could be considered a temporal filter while the latter a spatial filter. Temporal filter could be implemented by computing the correlation coefficient of each component's time-series with the experiment protocol, and choosing a specified number of most correlating components. Spatial filter could be implemented by computing the maximum spatial overlap between each component map and the desired spatial mask, and again selecting the most overlapping components. Further, a combination of temporal and spatial filters could also be implemented for the best effect. In any case, the time-series of the chosen number of independent components are then selected as input to the classifier. From each component's time-series, the most recent 17 data points are taken as spatio-temporal input to the classifier.

General Linear Model (GLM) and Granger Causality Model (GCM) based Feature Extraction could be done as follows. In one embodiment, the feature selection is in two steps: 1) Selection of time-series for input based on GLM, 2) GCM computation for feature selection where the GLM is used for training and the GCM is then used for feature extraction.

Step 1: Univariate method for identifying initial clusters of activation.

First, conventional, univariate General Linear Model (GLM) is applied to the training data set to obtain the statistical parametric maps (SPMs). Clusters of activations (total number of clusters=C) are then chosen at a user specified statistical threshold (example: P=0.01). Average, maximum or median time-series of signals such as BOLD signals from these clusters are then extracted and then presented to the GCM to generate directed influences and their F-statistics as described. A matrix, hereafter called Directed Influence Matrix (DIM), of size (C×C), containing the logarithm of F-statistics of directed influences of each cluster with other clusters are determined as features for the pattern classifier. A corresponding matrix of coordinates of cluster centers, hereafter called the Cluster Location Matrix (CLM), of size (C×4) is also maintained internally. Note that the 3 rows of the CLM matrix indicate the (x,y,z) coordinates respectively, and the fourth row the length of the sides of the square or the radius of the rectangle indicating the extent of the cluster's activation. The CLM matrix does not need to be directly input to the pattern classifier during training or testing, but used to map the input time-series to the cluster locations, in order to maintain the same clusters during both training and testing.

Step 2: Granger Causality Modeling for feature extraction.

Granger Causality Modeling (Granger, Econometrica. 37, 424-438, 1969; Granger, J Econ Dyn Control. 2, 329-352, 1980) is a method originally developed in economics for causal interaction between multiple events from time-series data. Since then it has been applied in neuroscience research for analysing connectivity of neurons from their firing patterns, from local field potentials, from EEG data (see Seth, 2001). More recently, GCM has been applied in conjunction with Vector Autoregressive Models (VAR) and to fMRI data (Abler et al., 2005), (Roebroeck et al., Neuroimage. 25, 230-242, 2005), and to investigate directed influences between neuronal populations (Roebroeck et al., 2004). The strength of the method exists in its data driven nature and its non-reliance on a priori specification of a model. These factors distinguish it from other effective connectivity approaches that aim at testing or contrasting specific hypotheses about neuronal interactions. Instead, GCM defines the existence and direction of influence from information in the data. Temporal precedence information is exploited to compute Granger causality maps that identify voxels that are sources or targets of directed influence from other voxels in the brain.

One embodiment provides the use of GCM applied to feature extraction for pattern classification of brain states from fMRI data in real time. GCM enables both spatial and temporal analysis of time-series data, and employing it as a method of feature selection before applying the features to a pattern classifier such as a Support Vector Machine (SVM) or Hidden Markov Model (HMM) will reduce the large dimensionality of the data, and also provides a means to capture both spatial and temporal interactions between distributed brain regions at once.

GCM may be implemented as linear autoregressive models that predict the evolution of time-series. Univariate autoregressive models describe a single time-series in terms of linear combinations of the past values (lags) of the time-series. Multivariate vector autoregressive (VAR) models include lags of multiple time-series. To exemplify one embodiment, consider two fMRI time-series X₁(t) and X₂(t) of length T, from two selected regions of the brain. The temporal dynamics of the X₁(t) and X₂(t) can be described by a bivariate autoregressive model:

$$\begin{matrix}{X}_{1} ( t ) = {\sum }_{j = 1}^{o} {A}_{11 , j} {X}_{1} ( t - j ) + {\sum }_{j = 1}^{p} {A}_{12 , j} {X}_{2} ( t - j ) + {E}_{1} ( t ) \\ {X}_{2} ( t ) = {\sum }_{j = 1}^{p} {A}_{21 , j} {X}_{1} ( t - j ) + {\sum }_{j = 1}^{p} {A}_{22 , j} {X}_{2} ( t - j ) + {E}_{2} ( t )\end{matrix} \qquad [ 2 ]$$

where p is the maximum number of lags included in the model (the model order, p<T), A contains the estimated coefficients of the model, E₁ and E₂ are residuals for each time-series. If the variance of the prediction error E₁ (or E₂) is reduced by the inclusion of the X₂ (or X₁) terms in the first (or second) equation, then it is said that X₂ (or X₁) Granger-causes X₁ (or X₂). X₂ Granger-causes X₁ if all the coefficients in A₁₂ are significantly different from zero. This can be tested by performing a t-test or F-test of the null hypothesis that A₁₂=0, with the assumption that X₁ and X₂ are covariance stationary. The magnitude of the Granger causality interaction can be estimated by taking the logarithm of the F-static. This concept is extended to the multivariate case by, for example, estimating a multivariable VAR-model. In such a case, X₂ Granger-causes X₁ if knowing X₂ reduces X₁'s prediction error when the time-series of all other variables (brain regions) X₃ . . . X_{N} are also taken into account. Multivariate analysis can improve robustness of the GCM results. For example, in a system in which X₁ and X₂ are both influenced by X₃ but are otherwise independent, a bivariate model of X₁ and X₂ may wrongly indicate that there is causal relationship between X₁ and X₂. A multivariate model would not have such a false positive, as knowing X₁ would not predict X₂ in the context of X₃. Thus, the multivariate model is used in one embodiment to overcome these deficiencies.

Principal Component Analysis (PCA) and Real-time Independent Component Analysis (ICA) could be done as follows. In one embodiment, the feature selection comprises two steps: 1) PCA and then ICA. The combination of these two models can provide advantages over the use of a single mode.

In one embodiment, the feature selection comprise using Fisher Criterion (FC):

$${score}_{F} ( X ) = \frac{{( mean ( {X}_{1} ) - mean ( {X}_{2} ) )}^{2}}{var ( {X}_{1} ) + var ( {X}_{2} )}$$

Fisher Criterion is used to select voxels having higher discriminability for different emotional states compared to other voxels. This module reduces computational time and can make the classifier more robust in the presence of noisy signals, such as signals having a low signal-to-noise ratio or signals with significant overlap with, for example, signals from head movement or magnetic field distortions.

Data driven methods are not based on neurophysiologic considerations and do not take into account past knowledge about brain activations obtained through other methods such as lesion studies, electrophysiological studies and so forth. By including a human expert in the process of feature selection, the accuracy of brain state classification could be potentially improved.

Alternatively, the model driven methods include both the use of a specified experimental protocol useful for a certain type of data analysis and the knowledge of the experimenter based information from prior experiments.

Cognitive, emotional, action and perception tasks have a large knowledge-base of previously reported experiments. Meta-analysis of such studies contains information about the coordinates of brain activation for different mental tasks. This information is used, in an exploratory way, to delineate brain areas that may be involved in the brain states and thus need to be discriminated. This can occur during offline training of the classifier and its v-fold cross validation. Those delineations that produce the best performance of the classifier are selected by the model based classification for the real-time classifier. Also useful for model driven classification are the results of preliminary results of activation clusters from a univariate GLM analysis. One exemplary, but nonliminting embodiment of the implementation of a human expert's feature selection incorporates a graphical user interface (GUI) comprising the following functions:
Execution of univariate GLM on the training data set to identify clusters of activation;
An optional mouse, touch, haptic, or keyboard based input method to select slice-based 2-dimensional or whole brain 3-dimensional regions-of-interest (ROIs) as activation clusters and their associated time-series as input to the classifier. A variety of rectangular, circular, and irregular ROIs as 2D-ROI selection; and cuboidal, spherical and irregular 3D shapes in 3D-ROI selection;
Selection of past n time points (See FIG. 2) for sliding window temporal input for the activation clusters;
Additional application of data-driven feature extraction methods (PCA and ICA); and
A data dimension guide that automatically computes the size of the spatio-temporal data for the training data set.

Using the above functions, the expert can work out by repeated explorations the dimensions for the training data set that produces a desirable level of accuracy of classification. The same spatiotemporal dimensions may also be applied during the real-time, online pattern classification.

Spatiotemporal pattern classification, also known as pattern recognition, occurs after feature extraction and is performed by a pattern classification module. Any pattern classifier that is able to classify brain states may be used. Pattern classification is the act of taking in raw data and taking an action based on the category of the data. Generally, pattern classification classifies data based on either a priori knowledge or on statistical information extracted from the patterns. Both a priori knowledge and statistical information may be used.

In one embodiment, spatiotemporal pattern classification is implemented using the Hidden Markov Model (HMM) as applied to the data. In another embodiment, spatiotemporal pattern classification module comprises a Support Vector Machine (SVM). Both these methods are known in the machine learning literature. However, their application to real-time spectroscopic imaging classification as described herein encompasses both spatial and temporal patterns of network of brain activity.

The use of the Hidden Markov Model (HMM) will reduce the large dimensionality of the data, and also provides a means to capture both spatial and temporal interactions between distributed brain regions at once.

The HMM is known in the art, (see Learning Hidden Markov Model Structure for Information Extraction, by Kristie Seymore, Andrew McCallum, and Roni Rosenfeld, American Association for Artificial Intelligence 99, Workshop on Machine Learning for Information Extraction, 1999.) The HMM is a statistical model in which the system being modeled is assumed to be a Markov process with unknown parameters which are determined from the observable parameters and is a dynamic Bayesian network. The use of the HMM model is illustrated in Example 2.

The use of a Support Vector Machine (SVM) is also contemplated. A SVM classifier can perform classification tasks base on real valued function approximation utilizing regression estimation. SVMs non-linearly map their n-dimensional input space into a high dimensional feature space where a linear classifier is constructed. The SVM is described in “A Tutorial on Support Vector Machines for Pattern Recognition”, by C. Burges, Data Mining and Knowledge Discovery, 2(2), 1-47, 1998, Kiuwer Academic Publisher, Boston, and on the web at aya.tecnion.ac.il/karniel/CMCC/SVM-tutorial.pdf.

Modified forms of the HMM classifier or the SVM classifier as well as other classifiers as known to one of ordinary skill in the art may also be used.

For embodiments when BOLD intensity values are used for the classification, in order to enable pattern classification in space, the BOLD intensity values from all the voxels of the brain (M×N×S voxels) may be provided as input to the pattern classifier for each time point. In order to enable pattern classification in time, BOLD intensity values from each voxel, from a sliding window of the past time points (or lags), are provided as input to the classifier. The pattern classifier would then work out the encoding both in space and time for a given task.

In one representative embodiment, the classification task is based on two separate sets of training data and testing data containing several instances of data. Each instance in the training set in this embodiment contains one target value, called the class label and several attributes or features. The goal of the classifier is to produce a model which predicts target values in the testing set when the attributes are given. In another representative embodiment, the classification task is based on three separate sets of training data and testing data containing several instances of data.

After the spatial and temporal data has been classified, information about the data is provided to either the subject or experimenter. The read-out may be provided to the subject as feedback, which may be used by the subject to direct voluntary control of one or more functions. Alternatively, the signal read-out may be used by the experimenter to determine the changes in brain states of the subject.

The signal read-out of the current brain state is presented to the subject or experimenter by different modalities, including, acoustic and visual, and is generalized to and not restricted to the representative embodiments as described below. The read-out may be provided for a single brain state. Alternatively, it may be provided for 2, 3, 4, 5, 6, 7, 8, 9, 10, or more brain states.

In one representative embodiment, the signal read-out device is in the form of visual read-out, such as changing bars of a graphical thermometer. Graphical thermometer may be used as a mode of feedback for fMRI-BCI and neurofeedback (Cana et al 2007, Sitaram et al 2007). This form of read-out may be used to represent the changing BOLD levels in a specific brain region of interest (ROI). However, the application of the graphical thermometer to the changing brain states and their intensities may be extended to include other information in the read-out.

In another embodiment, signal read-out can be presented as graphical iconic representations of different brain states or functions (for example, an icon for left hand and right hand motor imagery). The intensity or strength of a brain state (for example, the arousal level of the emotion state) can then be shown by a graphical thermometer beside the icon. Transitions from one brain state to another can be shown by arrows leading from the source state to the destination state. The advantage of the iconic method is that transition from one brain state to multiple other brain states can be presented as feedback, as against two brain states that can be presented by the graphical thermometer.

In one embodiment, the read-out comprises signal feedback such that the subject observes or hears the read-out signal and acts according to the changing signal. In another embodiment, the read-out comprises a signal such that a person observing the subject, either in real time or using a recording, can observe or hear the read-out signal.

The read-out is updated or refreshed at an interval that depends on the time involved for image acquisition and processing, based on the computational resources available and the efficiency of the algorithms with which they are implemented, thus directly affecting the performance of the system. A short interval is typically used for learning voluntary control of brain activity. For example, the read-out may be updated every 0.5 s or less, about every 1.0 s, about every 1.5 s, about every 2.0 s, or about every 3.0 s. In one embodiment, the feedback is updated about every 1.5 s or less.

### EXAMPLES

The following examples are included to demonstrate representative, non-limiting, embodiments. However, those of skill in the art should, in light of the present disclosure, appreciate that many changes can be Made in the specific embodiments which are disclosed and still obtain a like or similar result without departing from the spirit and scope of the disclosed material.

### Example 1

### Support Vector Machine (SVM) for Spatiotemporal Classification of Brain States

The SVM classification problem involves determining a vector Yₜ from a measurement matrix Xₜ. For classification of the fMRI data, matrix Xₜ represents the BOLD values at all the voxels in the brain for time points specified by t=1 to T, where T is the total number of time points, and yₜ is the experimental value of the task for each of those time points (FIG. 2(a)). For each time point t, Yₜ represents the type of task the subject was performing at each time point. For example in an experiment to discriminate between left hand and right hand motor imagery, each value of vector Yₜ may take on the following values: left hand imagery (class=1) and right hand imagery (class=−1). During the experimental procedure for collecting training data (for the example experiment above, when the subject performs alternating trials of left hand and right motor imagery), pre-processed BOLD values from all voxels in the brain for the time t=1 to T are collected as the matrix Xₜ, and each element of the vector Yₜ is marked as +1 or −1 according to the type of task, when the classification task is binary. Although, the current explanation uses binary classification as an example, the method explained is not restricted to binary classification, and can be readily extended to multiclass classification.

In the formulation of the SVM, the input matrix Xₜ is mapped to a high dimensional feature space, Zₜ, through a non-linear transformation function, g(·) so that Zₜ=g(Xₜ). The SVM algorithm attempts to find a decision boundary or a separating hyperplane in the feature space, given by the decision function:

D(Zₜ)=(W·Z)+w₀,

where W defines the linear decision boundaries. The solution W that represents the hyperplane can be obtained by solving for the equation:

yₜ[(W·Z)+w₀]≧1

The solution is optimal when ∥W∥²+C·f(ξ) is minimised under this constraint, where the parameter C>0, termed the regularisation constant, is chosen by the user. A large value of C corresponds to higher penalty for errors. The classification accuracy of the SVM model is tested first and developed using a V-fold cross-validation procedure. Starting from the linear kernel, different kernels could be explored to arrive at a suitable system that suits fMRI data and its nuances.

### Example 2

### Hidden Markov Model (HMM) for Spatiotemporal Classification of Brain States

The HMM could be seen as a finite state automaton, containing s discrete states, emitting an observation vector (or output vector) at every time point that depends on the current state. Each observation vector is modelled using m Gaussian mixtures per state. The transition probabilities between states are defined using a transition matrix. A detailed description of the method can be found in (Rabiner, 1989b).

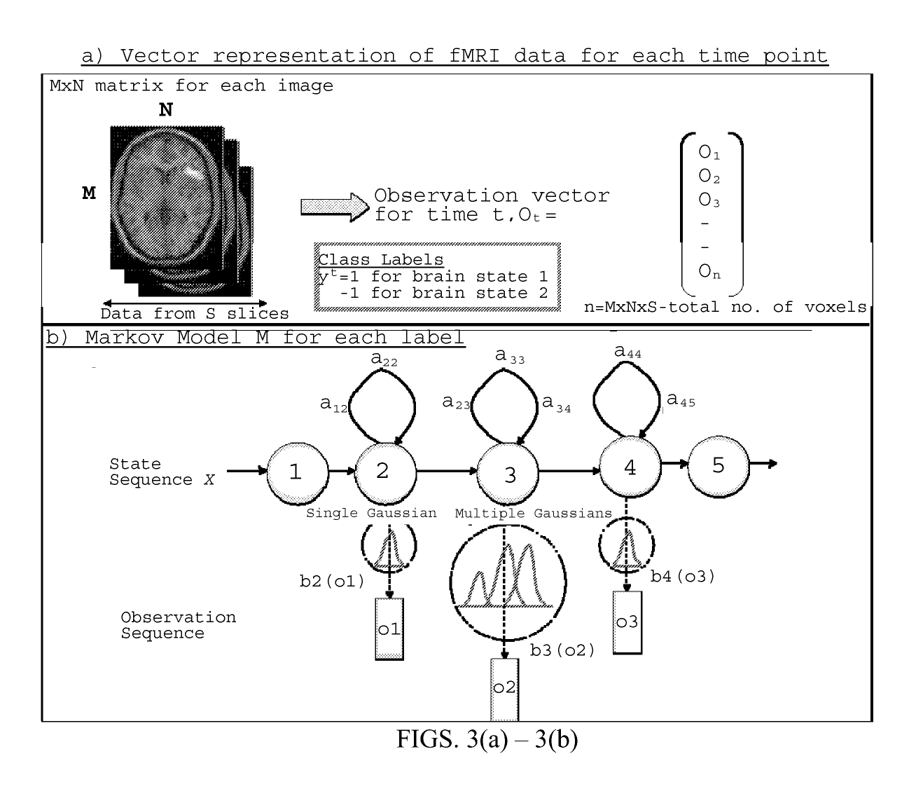

FIGS. 3(a) and (b) show schematic diagrams representing fMRI data and the HMM model for recognizing brain states. Each distinct type of brain state to be classified has to be modelled separately as depicted, and the model parameters have to be subsequently estimated separately with respective data. The HMM is designed as a left to right model, transitions being allowed from a state to itself and to any right neighbour state. Arrows from left to right indicate the allowed transitions of states. Beginning and end states are non-emitting, meaning that they do not result in any observations. Observations are modelled using a single Gaussian or a mixture of Gaussians.

In the HMM model denoted by M at each time t that a state j is entered, an observation vector oₜ is generated from the probability density bⱼ(oₜ). Further, transition from state i to state j is also probabilistic and is governed by the discrete probability aᵢⱼ. The joint probability O generated by the model M moving through the state sequence X is calculated as the product of transition probabilities and output probabilities. For the state sequence X in FIG. 2(b),

P(O|M)=a12b2(o1)a22b2(o1)a33b3(o2)

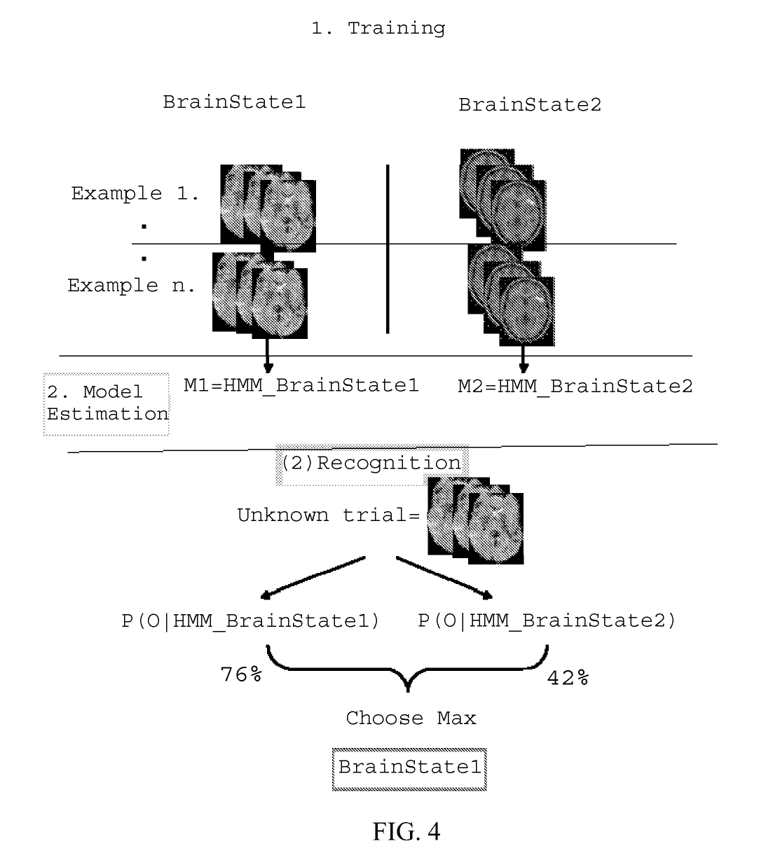

The observation O is known and the state sequence X is hidden. Given that X is unknown, the likelihood is computed by summing over all possible state sequences X=x(1), x(2), x(3), . . . , x(T), that is

$$P ( O , X \vert M ) = {\sum }_{x} {a}_{x ( o ) x ( 1 )} {\prod }_{t = 1}^{T} {b}_{x ( t )} ( {o}_{t} ) {a}_{x ( t ) x ( t + 1 )}$$

where x(o) is constrained to the model entry state and x(T+1) is constrained to the model exit state. Using the training samples different brain states or mental tasks, the parameters, {aᵢⱼ} and {bⱼ(o)}, of the model for the respective task/state are determined by an estimation procedure. To determine the parameters of the HMM model, the HTK uses the Baum-Welch re-estimation procedure (Young et al., 1993). To recognize an unknown trial data, the likelihood of each model generating the trial data (observation vector) is calculated using the Viterbi algorithm (Rabiner, 1989a), Rabiner and Juang, 1993), and the most likely model identifies the data as resulting from the distinct brain states (class=1 or −1). See, for example, FIG. 4. The method can be extended to classify more than 2 brain states. For example, it can be used to classify 3, 4, 5, 6, 7, 8, 9, 10, 11, 12, 13, 14, 15, about 20, about 30, about 40 or more brain states.

### Example 3

### Independent, Real-Time Detection and Neurofeedback of Multiple Emotional States in the Brain Using fMRI

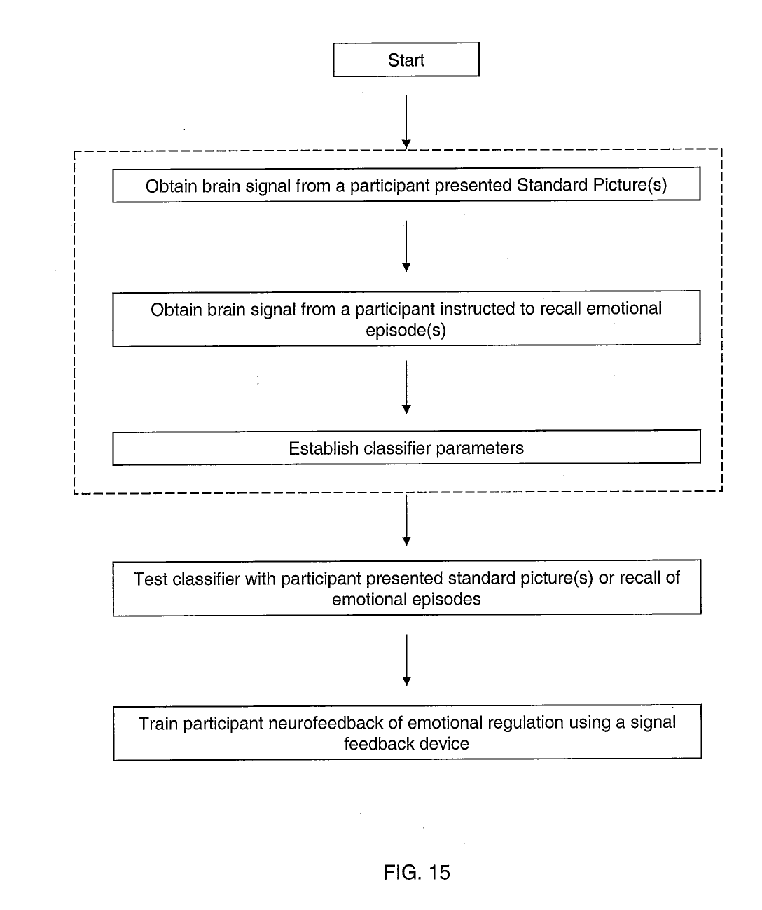

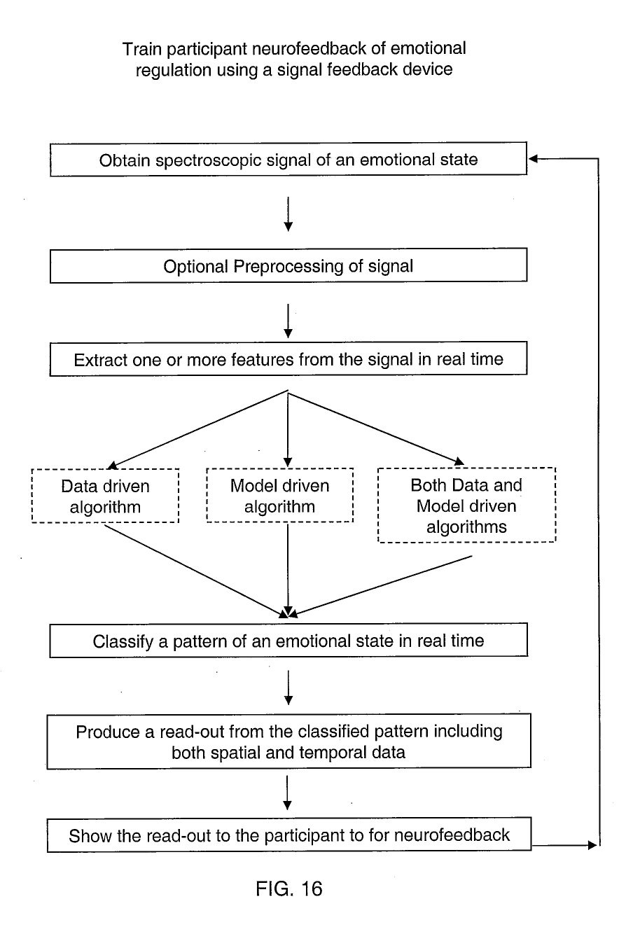

A real-time brain state detection system based on the support vector machine (SVM) classification of whole-brain fMRI signals for each repetition time of acquisition (TR) has been developed. The experiment was conducted in 3 stages, as exemplified in FIG. 15:
Stage 1: Acquisition of fMRI signals when participants are presented: a) Standard pictures from International Affective Picture System (IAPS) known to elicit discreet emotions: neutral, happy and disgust emotions. b) instructions to recall emotional episodes belonging to categories: neutral, happy and disgust. Classifier parameters are then estimated for each participant by using a custom developed SVM toolbox for fMRI data.
Stage 2: Testing the classifier for each participant on new fMRI data acquired during a similar paradigm (as above) involving viewing of emotional pictures or recall of emotions.
Stage 3: Neurofeedback training of emotional regulation using a thermometer feedback based on real-time brain state classification. The use of the feedback and processing used therein are exemplified in FIG. 16.

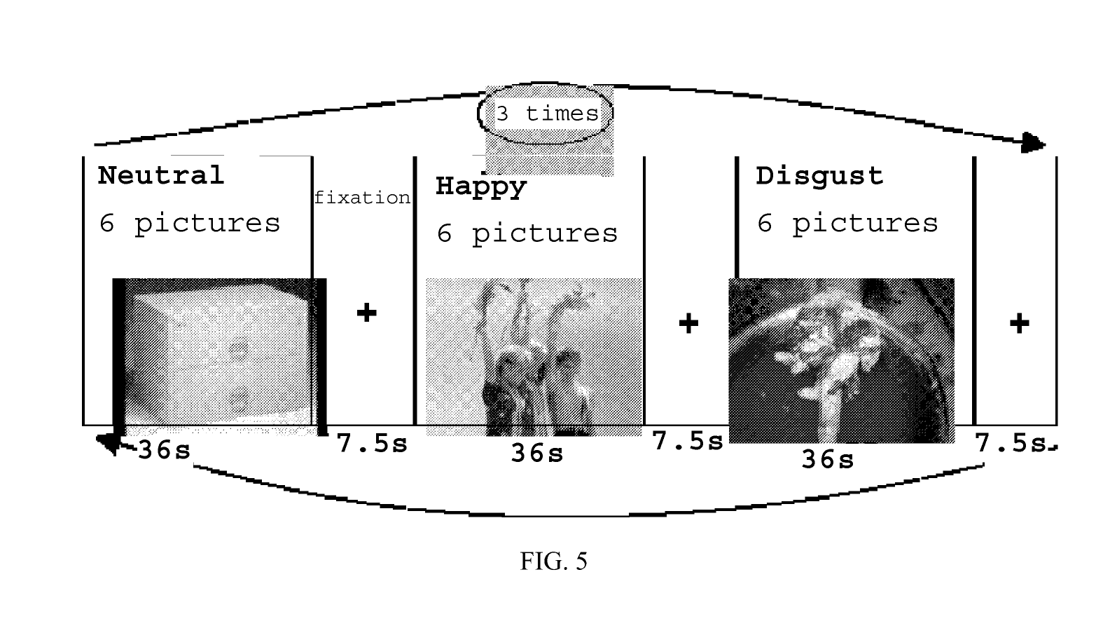

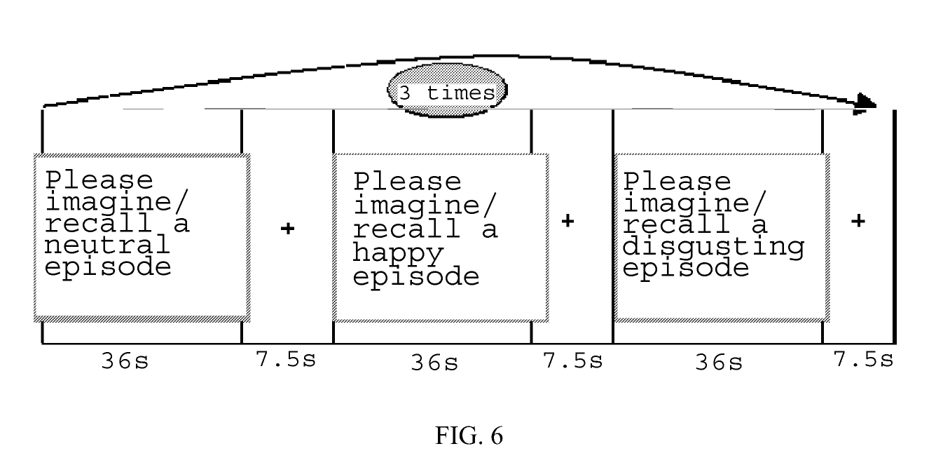

FIGS. 5 and 6 show the experimental paradigm for emotional picture presentation and instructions for recall of emotional episodes.

FMRI data acquired during the above experimental paradigm from each participant was corrected for head motion in real-time by performing realignment and smoothed to reduce noise. To reduce the large dimension of input to the classifier due to large number of voxels in the brain (64×64×16=about 65,000), a feature extraction step was performed. First, non-brain voxels were excluded with a thresholding method. Secondly, the Fisher Criterion (FC) (as shown in equation below) was used to select voxels which had higher discriminability for different emotional states compared to other voxels.

Classification was performed as described in the prior sections.

Classification was based on multivariate SVM to investigate which brain areas are associated in discriminating between different emotional states. SVM was used to discriminate between the following emotional states: neutral vs. happy, neutral vs. disgust, and happy vs. disgust. In addition, 5-fold cross validation was performed by dividing the whole data set into 5 separate permutations of a training set and a testing set, to each time test the accuracy of classification. In this process, the threshold of FC was selected which showed the lowest error rate and the lowest standard deviation of error.

A module was developed to determine the discriminability of each voxel as obtained by the SVM method. This module was defined as the ratio of mutual information between a weighted voxel value and the designed label to the mutual information between the weighted sum of other voxels without the voxel in question and the designed label (as shown in the equation below). The weighting used in the above definition is obtained from the weight vector obtained due to SVM model training.

MI(wᵢxᵢ;y)/MI(w₋ᵢx₋ᵢ;y)

where, w₋ᵢx₋ᵢ is the weight vector multiplied by the voxel vector for the i-th component. The predicted output of SVM is ŷ=sgn(wx+b).

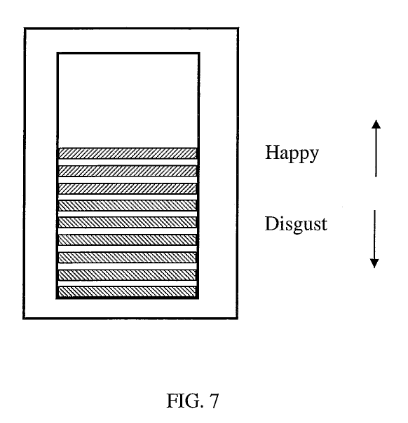

To test the reliability of the SVM classification on new data, subjects were shown new sets of IAPS pictures invoking the different emotional states, and fMRI signals were collected in real-time using a custom-written Echo Planar Imaging (EPI) sequence developed with real-time acquisition (1.5 s interval) and feedback for an fMRI Brain-Computer Interface. The real-time SVM classifier then determined the emotional state of the brain. Based on the classification result, a visual feedback was provided to the subject in the form of graphical thermometer with changing bars (FIG. 7). This is further exemplified in FIG. 16.

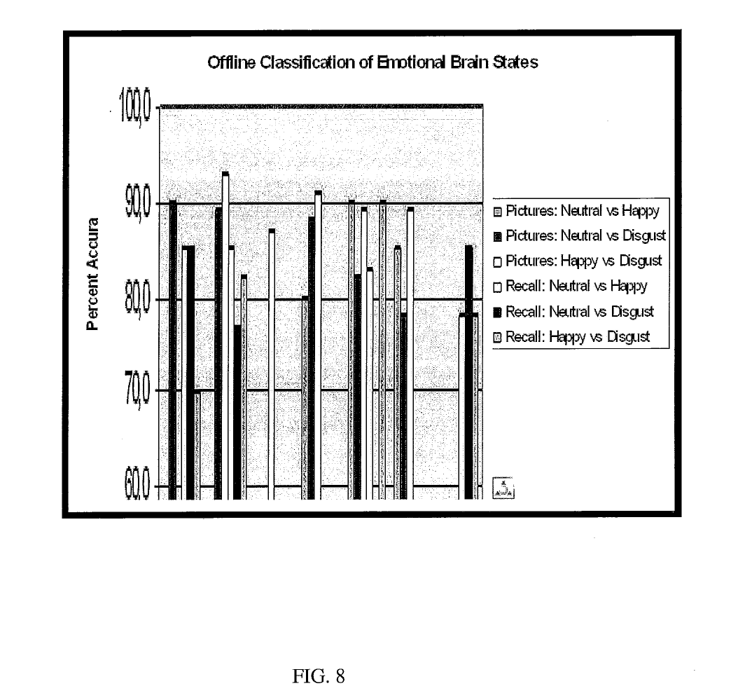

The SVM offline pattern classifier classifies brain states from the participants with an average accuracy of around 90% (FIG. 8), while the real-time version tested on 4 participants showed an average accuracy of above 70% and maximum accuracy of 90%. Remarkably, on a few participants, a classifier that was trained to discriminate emotional states elicited by pictures could also discriminate emotional states realized through recall with about ˜70% accuracy. This result suggests that there are common areas of activation between picture-based and recall-based emotion induction.

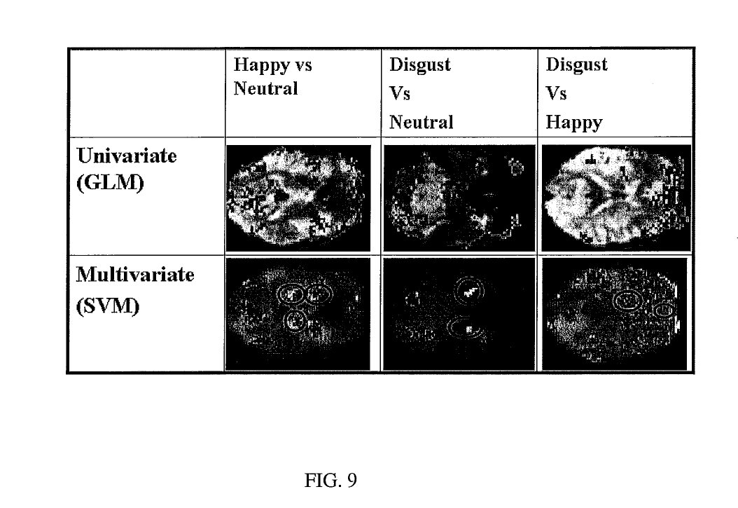

The activation patterns obtained using the univariate (GLM) method and realtime multivariate (SVM) methods of analysis were compared. FIG. 9 shows the difference of activations between the two methods. With the GLM maps, gray voxels show significant activations at the given contrast (e.g., Happy v. Neutral). With the SVM maps, gray voxels show significant voxels that discriminate between the emotional states. Notice the different activation clusters that SVM uses for discrimination of emotional states. These areas might be coding the spatiotemporal information representing the brain state in a way that the voxel-wise GLM is not sensitive enough to detect.

Using the methods, systems, and apparatus described multiple emotional states elicited in an individual's brain (either by viewing emotional pictures or by recall of emotional episodes) can be recognized and discriminated in real-time from fMRI signals. BCI can be developed using a pattern classifier to provide feedback from an entire neural network involved in a cognitive/emotional activity in contrast to previous single ROI methods. Differences between multivariate SVM based methods and univariate GLM based methods in identifying discriminating voxels between brain states are shown.

The application of real-time classification of emotion states in the brain from spectroscopic signals, and subsequently using the classification to provide feedback information to the subject to modulate brain activity to enhance or reduce the effect or intensity of the brain state is shown.

While this example describes three discrete emotional states, the method applies generally together discrete and complex emotional states including, but not limited to

anxiety, acceptance, anger, anticipation, disgust, distress, depression, desire, fear, happiness, interest, joy, panic, relaxation, rage, sadness, shame, sorrow, and surprise. Further, this example describes classification training based on emotion inducing pictures or recall of personal emotional episodes, but is equally applicable to emotions induced by auditory, audio-visual, olfactory, gustatory, touch and pain stimuli.

Having described various embodiments with reference to particular compositions, theories of effectiveness, and the like, it will be apparent to those skilled in the art that it is not intended that there should be a limitation to such illustrative embodiments or mechanisms, and that the modifications can be made without departing from the scope or spirit, as defined by the appended claims. It is intended that all modifications and variations be included with the scope of the described embodiments. The claims are meant to cover the claimed components and steps in any sequence that is effective to meet the objectives there intended, unless the context specifically indicates the contrary.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs miming on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

All references, including but not limited to patents, patent applications, and non-patent literature are hereby incorporated by reference herein in their entirety.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The following acronyms are used in the present application:
ANOVA analysis of variance
BCI brain-computer interface
BOLD blood-oxygen-level dependent response, which is the vascular response to neural activity
CLM cluster location matrix
DIM directed influence matrix
DIM directed influence matrix
DTI diffusion tensor imaging
DWI diffusion-weighted imaging
EEG electroencephalography
EPI echo planar imaging
fMRI functional magnetic resonance tomography
FPA fixed point algorithm
GCM granger causality model
GLM general linear model
HMM hidden Markov model
ICA independent component analysis
MNN multilayer neural networks
NIRS near infrared spectroscopy or near infrared spectroscope
OT optical topography
PCA principal component analysis
ROI regions of interest
SNR signal-to-noise ratio
SPM statistical parametric map
SVM support vector machines
VAM vector autoregressive models
VAR (multivariate) vector autoregression

We claim:

1. An apparatus comprising:
a real-time spatiotemporal pattern classifier configured to classify spatial and/or temporal spectroscopic data of brain states in real time, which includes
a feature extraction module and
a pattern classification module; and
a read-out having spatial and/or temporal components.

2. The apparatus of claim 1, wherein the real-time spatiotemporal pattern classifier is configured to classify both spatial and temporal spectroscopic data and the read-out has both spatial and temporal components.

3. The apparatus of claim 1, wherein the feature extraction module comprises a data driven algorithm or a model driven algorithm.

4. The apparatus of claim 3, wherein the data driven algorithm comprises a principal component analysis (PCA) model.

5. The apparatus of claim 3, wherein the data driven algorithm comprises an independent component analysis (ICA) model.

6. The apparatus of claim 3, wherein the data driven algorithm comprises a general linear model (GLM) and a Granger causality model (GCM).

7. The apparatus of claim 1, wherein the pattern classification module comprises a Hidden Markov Model (HMM) or a Support vector machine (SVM).

8. The apparatus of claim 1, wherein the real-time spatiotemporal pattern classifier is configured to classify data in about 0.5-3.0 seconds.

9. A system comprising:
a spectroscope;
a real-time spatiotemporal pattern classifier configured to classify spatial or temporal data in real time, which includes
a feature extraction module and
a pattern classification module; and
a read-out having spatial and/or temporal components.

10. The system of claim 9, wherein the spetroscope is a functional MRI device (fMRI).

11. The system of claim 9, wherein the spectroscope is a near infrared spectroscope (NIRS).

12. The system of claim 10, wherein the fMRI is configured to utilize an echo planer imaging sequence.

13. A method comprising:
obtaining a spectroscopic signal from a subject brain, the signal having spatial or temporal data;
extracting one or more features from the signal in real time;
classifying a pattern of a brain state in real time; and
generating a read-out comprising said spatial and/or temporal data.

14. The method of claim 13, wherein two or more brain states are classified in real time.

15. The method of claim 13, wherein the read-out is associated with a specific physical, cognitive, or emotional task of the subject.

16. The method of claim 13, further comprising providing feedback to the subject.

17. The method of claim 13, further comprising recognizing a brain state in the subject brain.

18. The method of claim 13, further comprising recognizing, diagnosing, or treating a disease or condition in the subject.

19. The method of claim 18, wherein the condition is the utterance of a false statement.

20. The method of claim 18, wherein the condition is reduced limb function.
